(19) United States
(12) Patent Application Publication
(10) Pub. No.: US 2024/0130730 A1
(43) Pub. Date: Apr. 25, 2024

(54) KIT FOR TREATING DAMAGED NERVES

(71) Applicant: Reddress Ltd., Pardes Hana (IL)

(72) Inventor: Alon Kushnir, Givat Ada (IL)

(21) Appl. No.: 18/277,511

(22) PCT Filed: Feb. 22, 2022
(86) PCT No.: PCT/IL2022/050206

§ 371 Date: Aug. 15, 2023

(30) Foreign Application Priority Data
Feb. 23, 2021 (IL) 281044

(51) Int. Cl. A61B 17/11; A61L 27/38

(52) U.S. Cl. CPC A61B 17/1128; A61L 27/383; A61L 27/3834; A61B 2017/00884

(57) ABSTRACT

The present disclosure provides a method and a kit for inducing growth or regeneration of a damaged nerve of a subject, or the treatment thereof by applying a clot of blood that is withdrawn from the subject. The blood that is withdrawn from the subject is introduced to a lumen of a nerve enveloping hollow element that envelopes the damaged portion of the nerve while controllably coagulating within the lumen to form a clot on the damaged portion of the nerve. The clot, while in physical contact with the damaged portion of the nerve, enhances the rehabilitation of the nerve and may partially or fully restore its functionality.

### TECHNOLOGICAL FIELD

The present disclosure is in the field of medical treatment of damaged nerves.

### BACKGROUND ART

References considered to be relevant as background to the presently disclosed subject matter are listed below:
WO 2019/058373
WO 2019/058375
U.S. Pat. No. 9,180,142

Acknowledgement of the above references herein is not to be inferred as meaning that these are in any way relevant to the patentability of the presently disclosed subject matter.

### GENERAL DESCRIPTION

The present disclosure provides a method and a kit for inducing growth or regeneration of a damaged nerve of a subject, or the treatment thereof by the use of whole blood that is withdrawn from the subject and coagulated in situ in a controlled manner. The nerve that is treated may be a crushed nerve, a nerve damaged by a certain disease process (e.g., inflammation), a partially cut or a fully cut nerve that leaves two nerve stumps. By some embodiments the blood that is withdrawn from the subject is introduced into a lumen of a nerve-enveloping sleeve that envelopes the damaged portion of the nerve or the two end portions of opposite nerve stumps that are brought into within the sleeve from opposite ends thereof, while controllably coagulating within the lumen to form a clot or coagulated blood matrix on such damaged nerve portions and/or between two cut nerve stumps. The clot, while in physical contact with the damaged portion of the nerve, enhances the rehabilitation of the nerve and may partially or fully restore its functionality. By other embodiments use is made of a nerve graft to bridge the two stumps of a cut nerve.

The term “nerve treatment portion” or “treatment portion” will be used herein to denote a portion of the nerve that is treated in accordance with this disclosure to induce nerve regrowth across a damaged nerve portion, between two end portions of stumps of cut nerves, or a portion that includes two end portions of cut nerve stumps with a nerve graft between the two.

Whole blood that is used in this disclosure, while typically blood withdrawn from the individual, may also be blood obtained from a donor or from a blood bank. Thus, the term “blood” or “whole blood”, if not specifically stated otherwise, should be interpreted as meaning whole blood regardless of its source.

The term “coagulation inducer(s)” is used herein to denote one or more coagulating agents and/or anti-anti-coagulants. The term “mixture” is used herein to denote a mixture that comprises whole blood and a coagulation inducer(s).

The term “clot”, “blood clot” or “coagulated blood” are used interchangeably to denote a coagulated whole blood matrix formed from said mixture.

In the following, where a method is described, the order of steps in the performance of the method may or may not be in the order in which they are written.

The subject treatable in accordance with this disclosure may be a human or a non-human animal.

Thus, an aspect of the present disclosure provides a method for regenerating a nerve in or across a nerve treatment portion. The method comprises fitting a sleeve having a lumen to envelope the nerve treatment portion such that it is accommodated within said lumen. The method also comprises the introduction of a mixture of whole blood with said coagulation inducer(s) (the mixing being carried out prior or during the introduction) into said lumen. The nerve-enveloping sleeve may be made or configured to envelope the nerve treatment portion while maintaining a space between its inner face and the nerve treatment portion to allow introduction of coagulating blood to the space to form a blood clot on the damaged nerve. The mixture of whole blood with the coagulation inducer(s) is typically introduced into said lumen prior to complete coagulation of the mixture.

It is to be noted, for any aspect of the present disclosure, that the whole blood may be withdrawn from a subject and initially mixed with anti-coagulating agents to prevent its natural coagulation. At a desired timing, the whole blood is mixed with the coagulation inducer(s) to controllably coagulate the whole blood on the damaged nerve portion to form the desired blood clot thereon.

In some embodiments of the method, said fitting comprises placing a film or sheet over the nerve treatment portion and enveloping said portion with said sheet to form the nerve enveloping sleeve. In other embodiments the sleeve may be an a priori tubular element and the treatment portion is introduced therein. The sleeve may be made of a plastic material or any other suitable and biocompatible material.

It is to be noted that any combination of the described embodiments with respect to any aspect of this present disclosure is applicable. In other words, any aspect of the present disclosure can be defined by any combination of the described embodiments.

Yet another aspect of the present disclosure provides a method for inducing growth of nerve fibers across a damaged nerve portion. The method comprises fitting a nerve-enveloping sleeve having a lumen, such as a nerve guidance conduit to envelope said nerve portion such that it resides within said lumen; and introducing said mixture into said lumen prior to its complete coagulation of the mixture.

In some embodiments of the method, the sleeve is a nerve guidance conduit.

In some embodiments of the method, the damaged nerve portion comprises cut nerve fibers.

In some embodiments of the method, the nerve portion includes two cut nerve stumps that are surgically joined or brought into proximity within the sleeve.

In some embodiments of the method, said fitting comprises placing a film or sheet over the treatment portion and enveloping said portion with said sheet to form said conduit.

In some embodiments of the method, said fitting comprises introducing the two nerve stumps into said sleeve, one from each end thereof; and bringing the two stumps into proximity to one another within said nerve enveloping sleeve. The mixture may be made, by some embodiments, to fill the space between the two stumps.

In some embodiments of the method, said bringing comprises securing the two stumps within said sleeve by suturing.

In some embodiments of the method, said introducing comprises filling a portion of the conduit's lumen with the mixture such that it comes into contacts with said nerve portion.

In some embodiments of the method, the nerve portion comprises two cut nerve stumps and said introducing is carried out such that said mixture comes into contact with said stumps.

In some embodiments, the method further comprises saturating the blood, prior to said introducing, which may be before, during or after mixing the blood with a coagulating agent, with an oxygen-rich gas, e.g., oxygen-enriched air or pure oxygen.

In some embodiments, the method further comprises mixing the blood, prior to said introducing, with one or more autologous cells. In some embodiments, said one or more autologous cells comprise Schwann cells.

Yet another aspect of the present disclosure provides a method for facilitating nerve regeneration between first and second nerve stumps of a cut nerve. By one embodiment, the method comprises fitting and securing, e.g., by suturing, over said first nerve stump a first open end portion of a nerve guidance conduit, and fitting and securing over said second nerve stump a second open end portion of said nerve guidance conduit. The nerve guidance conduit defines a lumen extending between said first and second open end portions. The method further comprises mixing whole blood with a coagulation inducer(s) and introducing the mixture into said lumen prior to its complete coagulation, namely while it is still in a flowable form, and permitting the blood to coagulate within said lumen.

In some embodiments of the method, said securing comprises suturing said first and second nerve stumps to the first and second open end portions, respectively.

In some embodiments of the method, said introducing comprises filling a portion of the lumen with blood such that it contacts both first and second nerve stumps, namely filling the gap or space between the first and second nerve stumps with blood and creating a continuous matrix of nerve stumps and blood.

In some embodiments, the method further comprises saturating the blood, prior to said introducing, with oxygen.

In some embodiments of the method, the anti-anti-coagulant is one or more from a group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate (“first group”).

In some embodiments of the method, the one or more coagulation agents or anti-anticoagulating agents is one or more of the group consisting of: Kaolin, Ca′, e.g. in the form of calcium salts such as Calcium Gluconate, Mg′, negatively charged phospholipid (PL) and protamine sulfate (“second group”); or a mixture of one or more of the group of the first group with one or more of the second group.

In some embodiments, the method further comprises, prior to said introducing, adding to or saturating the blood with keratin, stem cells (of all types), growth factors, collagen, bone marrow or other gases or materials.

In some embodiments of the method, said introducing comprises inserting a blood applicator into the lumen and injecting the blood into the lumen by said blood applicator, e.g., a syringe.

In some embodiments, the method further comprises mixing the blood, prior to said introducing, with one or more autologous cells. In some embodiments, said one or more autologous cells comprise Schwann cell.

In some embodiments of the method, said conduit is made of or of comprises collagen, polyglycolic acid (PGA), polycaprolactone (PCL), polyvinyl alcohol (PVA), and porcine small intestine submucosa (SIS).

Yet another aspect of the present disclosure provides a kit for treating, inducing growth, or regenerating a damaged nerve portion of a subject, that may be employed in the above method. The kit comprising a nerve enveloping sleeve having a lumen for enveloping said damaged nerve portion such that said portion is within at least a portion of said lumen; one or more blood withdrawal devices for allowing withdrawal of blood from the subject; optionally, one or more blood collection receptacles for receiving the blood withdrawn from the subject or means for receiving whole blood obtained from a blood bank; a coagulation assembly configured for permitting mixture of the withdrawn blood with the coagulation inducer(s) for initiating coagulation process of the blood; and an applicator for introducing the incomplete coagulated blood into said lumen.

In some embodiments of the kit, the one or more collection receptacles are vacuum tubes.

In some embodiments of the kit, the coagulation assembly comprises a vessel for receiving the withdrawn blood, said vessel being configured for contacting the blood with said coagulation inducers.

In some embodiments, the kit further comprises one or more coagulation inducers(s) for mixing with the withdrawn blood.

In some embodiments of the kit, the one or more coagulation inducer(s) are comprised within said one or more blood collection receptacles.

In some embodiments of the kit, the anti-anticoagulation agent is selected from a group consisting of one or more of said first group, or one or more of said second group or a combination of one or more of said first group with one or more of said second group.

In some embodiments, the kit further comprises a film or sheet for placing over said damaged nerve portion for forming the nerve enveloping sleeve.

In some embodiments of the kit, said sleeve is a nerve guidance conduit.

In some embodiments of the kit, the nerve treatment portion is a cut nerve with a first and second nerve stumps.

In some embodiments, the kit further comprises securing or retaining elements for securing the nerve treatment portion to said sleeve.

In some embodiments, the kit is intended for use in a method for treating, inducing growth, or regenerating a damaged nerve portion. The method includes (i) enveloping said damaged nerve portion with said sleeve having a lumen such that said damaged nerve portion resides in said lumen; (ii) withdrawing whole blood from the subject; (iii) mixing the subject's blood with one or more coagulation inducers; (iv) prior to complete coagulation of the blood, introducing the blood with the coagulation agent into said lumen; and (v) permitting the blood to coagulate in said lumen.

In some embodiments, the kit is intended for use in a method according to any one of the above-described embodiments of the method.

By other aspects of this disclosure a bridging nerve graft is employed to bridge two stumps of a cut nerve. These aspects include a method, to be referred to as a “nerve graft aspect method” and a kit, to be referred to as a “nerve graft aspect kit”. According to the graft aspect method for inducing growth of nerve fibers or for regenerating a nerve between a first nerve stump and a second nerve stump, comprises:
(i) positioning the nerve graft in between the first and second nerve stumps. The nerve graft can be autologous or non-autologous nerve graft; it may be obtained from a donor or a cadaver; it may be allogeneic or xenogeneic. The nerve graft can be partially synthetic, processed human nerve allograft or fully synthetic. The nerve graft may at times be fresh, e.g. kept in an appropriate medium, may be frozen or preserved in another manner. The nerve graft may be treated before use to be made acellular, for example, by freezing. The nerve graft may be placed without suturing within lumen of the nerve-enveloping sleeve (e.g., a tube), before fitting it over one of the stumps or after such fitting. The nerve graft comprises empty endoneurial tubes, that are micro channels/tunnels left by the axons that previously past through them, allowing nerve growth therethrough between the first and second nerve stumps. The micro channels extend between the two ends of the nerve graft, namely extending between the first nerve stump and the second nerve stump when the graft is linking the stumps;
(ii) introducing a mixture of whole blood and a coagulation inducer into said channels prior to the complete coagulation of the mixture, said introducing is carried out prior or after said positioning. This may be achieved by forced introduction, by bringing one or both ends of the nerve graft into contact with said mixture and permitting the blood to enter into said channels by a capillary force, or by any other suitable means. This introduction may be before or after said positioning; and
(iii) permitting the blood to coagulate within said channels.

It is to be noted that at least a portion of the coagulated whole blood can come into contact with the first and/or the second nerve stumps. Thus, in some embodiments, the method further comprises forming a continuous coagulated whole blood matrix between the first nerve stump and the nerve graft and between the nerve graft and the second nerve stump, thereby linking the first and the second stumps via the nerve graft channels.

In some embodiments, the nerve graft aspect method further comprises securing said graft to one or both of said first and said second nerve stumps.

In some embodiments of the method, said securing comprises suturing said first and second nerve stumps to said nerve graft.

In some embodiments, the nerve graft aspect method further comprises saturating the blood, prior to said introducing, with oxygen.

In some embodiments, the nerve graft aspect method further comprises mixing the blood, prior to said introducing, with one or more autologous cells.

In some embodiments of the nerve graft aspect method, said one or more autologous cells comprise Schwann cell.

In some embodiments of the nerve graft aspect method, said one or more coagulation inducer(s) are in powder form, wherein at least a portion of the particles of said powder are of a size that fits to enter said channels. Namely, the particles are sufficiently small, so their diameter is smaller than the diameter of the cross-section of the channels.

In some embodiments, the nerve graft aspect method further comprises filtering said powder prior to mixing it with the blood to obtain a mixture with at least a portion of particles having a size less than 2.5 microns, less than 2, 1.5, or even less than 1 microns. In some embodiments, the powder includes more than 50% of the particles that are of size that fits to enter into said channels.

In some embodiments of the nerve graft aspect method, said filtering comprises: (i) introducing the powder into liquid for a selected amount of time, (ii) collecting floating particles, namely collecting a top portion of the liquid, and (iii) drying said particles to obtain desired filtered particles.

In some embodiments of the nerve graft aspect method, said coagulation inducer is one or more of said first group, or one or more of said second group or a combination of one or more of said first group with one or more of said second group.

In some embodiments, the nerve graft aspect method further comprising fitting said sleeve, i.e. a nerve guidance conduit, over at least one of: the first nerve stump and a portion of the nerve graft, the second nerve stump and a portion of the nerve graft or the first nerve stump, the nerve graft and the second nerve stump; wherein said introducing comprises filling said lumen with said mixture.

The nerve graft aspect kit for inducing growth or regenerating a damaged nerve portion of a subject having a first nerve stump and a second nerve stump comprises the elements of the kit described above and, in addition, also a nerve graft having micro channels allowing nerve growth therethrough or means for harvesting or producing such nerve graft.

In some embodiments of the nerve graft aspect kit, said one or more collection receptacles are vacuum tubes.

In some embodiments of the nerve graft aspect kit, the coagulation assembly comprises a vessel for introducing the withdrawn blood, said volume is contactable with one or more coagulation agents or anti-anticoagulation agents.

In some embodiments, the nerve graft aspect kit further comprises one or more coagulation inducer(s) for mixing with the withdrawn blood.

In some embodiments of the nerve graft aspect kit, the one or more anti-coagulation agents are comprised within said one or more blood collection receptacles.

In some embodiments of the nerve graft aspect kit, the coagulation inducer is one or more of said first group, or one or more of said second group or a combination of one or more of said first group with one or more of said second group.

In some embodiments, the nerve graft aspect kit further comprises said sleeve for placing over at least one of: the first nerve stump and a portion of the nerve graft, the second nerve stump and a portion of the nerve graft or the first nerve stump, the nerve graft and the second nerve stump. By using the applicator, the whole blood undergoing coagulation is intended to be introduced into a lumen of said sleeve. By doing that, the whole blood forms a continuous coagulated whole blood matrix between the first nerve stump and the second nerve stump via the channels of the nerve graft. The formed clot from the coagulating whole blood enhances the regeneration process of the nerve.

In some embodiments of the kit, said sleeve is a nerve guidance conduit.

In some embodiments, the nerve graft aspect kit is intended for use in the nerve graft aspect method.

In some embodiments, the nerve graft aspect kit is intended for use in any one of the above-described embodiments of the method that uses the nerve graft, or any combination thereof.

### Embodiments

The following are optional embodiments and combinations thereof in accordance with aspects of the present disclosure. These embodiments are intended to add to add to and not limit the disclosure herein as generally described above. In these embodiments, methods steps may or may not be in the order in which they are written.
1. A method for regenerating a nerve in or across a damaged nerve portion, comprising:
   fitting a sleeve to envelope said nerve portion such that it is accommodate in the sleeve's lumen; and
   introducing a mixture of whole blood, typically withdrawn from the subject (but may also be from a donor or a blood bank), with one or more coagulation inducers, which may be coagulating agents and/or anti-anti coagulating agents, into said lumen prior to its complete coagulation.
2. The method of embodiment 1, wherein said fitting comprises placing a film or sheet over said portion and enveloping said portion with said sheet to form said sleeve.
3. A method for inducing growth of nerve fibers across a damaged nerve portion, comprising:
   fitting a sleeve to envelope said nerve portion; and
   introducing a mixture of whole blood, typically withdrawn from the subject, with one or more coagulation inducers, into said lumen prior to its complete coagulation.
4. The method of any one of embodiments 1-3, wherein the damaged nerve portion comprises cut nerve fibers.
5. The method of embodiment 4, wherein said nerve portion includes two cut nerve stumps that are surgically joined or brought into proximity.
6. The method of embodiment 5, wherein said fitting comprises:
   placing a film or sheet over said portion and enveloping said portion with said sheet to form said sleeve.
7. The method of embodiment 5, wherein said fitting comprises:
   introducing the two nerve stumps into said sleeve, one from each end of the sleeve;
   bringing the two stumps into proximity to one another within said conduit.
8. The method of embodiment 7, wherein said bringing, comprises securing the two stumps within said sleeve by suturing.
9. The method of any one of embodiments 3-8, wherein said introducing comprises filling a portion of the sleeve's lumen with the mixture such that it comes into contacts with said nerve portion.
10. The method of embodiment 9, wherein said nerve portion includes two cut nerve stumps and said introducing is carried out such that said mixture comes into contact with said stumps to form a coagulated blood matrix between the stumps.
11. The method of any one of embodiments 1-10, comprising saturating the blood, prior to said introducing, with an oxygen-rich gas.
12. The method of any one of embodiments 1-11, comprising mixing the blood, prior to said introducing, with one or more autologous cells.
13. The method of embodiment 12, wherein said one or more autologous cells comprise Schwann cell.
14. A method for inducing growth of nerve fibers between a first and second nerve stumps of a cut nerve, comprising:
   fitting and securing over said first nerve stump a first open end portion of a sleeve, and fitting and securing over said second nerve stump a second open end portion of the sleeve, wherein said sleeve defining a lumen extending between said first and second, open, end portions;
   mixing whole blood withdrawn from the subject with one or more coagulation inducers and introducing it into said lumen prior to complete coagulation of the mixture;
   permitting the blood to coagulate within said lumen.
15. The method of embodiment 14, wherein said securing comprises suturing said first and second nerve stumps to the first and second end portions, respectively.
16. The method of embodiment 14 or 15, wherein said introducing comprises filling the lumen or a portion thereof with said mixture such that the mixture and a coagulated blood matrix formed therefrom contacts both first and second nerve stumps.
17. The method of any one of embodiments 14-16, comprising saturating the blood, prior to said introducing, with oxygen.
18. The method of any one of embodiments 14-17, wherein said introducing comprises inserting a blood applicator into the lumen and injecting the blood into the lumen by said blood applicator.
19. The method of any one of embodiments 14-18, comprising mixing the blood, prior to said introducing, with one or more autologous cells.
20. The method of embodiment 19, wherein said one or more autologous cells comprise Schwann cell.
21. A kit for inducing growth or regenerating a damaged nerve portion of a subject, the kit comprising:
   a sleeve having a lumen for enveloping said damaged nerve portion such that said portion resides in at least a portion of said lumen;
   optionally, one or more blood withdrawal devices for withdrawal of blood from the subject;
   one or more blood collection receptacles for receiving the blood;
   a coagulation assembly configured for permitting mixture of the withdrawn blood with one or more coagulation inducers for initiating a coagulation process of the withdrawn blood; and
   an applicator for introducing the incomplete coagulated blood into said lumen.
22. The kit of embodiment 21, wherein said one or more collection receptacles are vacuum tubes.
23. The kit of embodiment 21 or 22, wherein the coagulation assembly comprises a vessel for receiving the blood, said vessel being configured for contacting the blood with said coagulation inducers.
24. The kit of any one of embodiments 21-23, comprising one or more coagulation inducers.
25. The kit of embodiment 24, wherein the one or more coagulation inducers are comprised within said one or more blood collection receptacles.
26. The kit of embodiment 24 or 25, wherein the coagulation inducers are anti-anticoagulation agent selected from the group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate.
27. The kit of any one of embodiments 21-26, said one or more coagulation inducers are selected from: Kaolin, Ca 2±, Mg′, negatively charged phospholipid (PL) and protamine sulfate.
28. The kit of any one of embodiments 21-27, comprising a film or sheet for placing over said damaged nerve portion for forming said nerve enveloping sleeve.
29. The kit of any one of embodiments 21-27, wherein said nerve enveloping sleeve is a nerve guidance conduit.
30. The kit of any one of embodiments 21-29, comprising securing elements for securing said damaged nerve portion to the nerve enveloping sleeve.
31. The kit of any one of embodiments 21-30, wherein said damaged nerve portion is a cut nerve having a first and second nerve stumps.
32. The kit of any one of embodiments 21-31, for use in a method for treating, inducing growth, or regenerating a damaged nerve portion, the method comprising:
   (i) enveloping said damaged nerve portion with a sleeve having a lumen such that said damaged nerve portion is accommodated within said lumen;
   (ii) withdrawing whole blood from the subject;
   (iii) mixing the subject's blood with one or more coagulation inducers;
   (iv) prior to complete coagulation of the blood, introducing the blood with the coagulation agent into said lumen; and
   (v) permitting the blood to coagulate within said lumen.
33. The kit of any one of embodiments 21-31, for use in a method according to any one of embodiments 1-20.
34. A method for inducing growth of nerve fibers between a first nerve stump and a second nerve stump, comprising:
   placing a nerve graft between the first and second nerve stumps, optionally attaching each of the first and second nerve stumps to a different end of the nerve graft, said nerve graft comprising micro channels allowing nerve growth therethrough between the first and second nerve stumps;
   introducing a mixture of whole blood, typically blood withdrawn from the subject, with one or more coagulation inducers, into said channels prior to the complete coagulation of the mixture, said introducing is carried out prior or after said placing;
   permitting the blood to coagulate within said channels.
35. The method of embodiment 34, comprising securing said graft to one or both of said first and second nerve stumps.
36. The method of embodiment 35, wherein said securing comprises suturing said first and second nerve stumps to said nerve graft.
37. The method of any one of embodiments 34-36, comprising saturating the blood with oxygen, prior to said introducing.
38. The method of any one of embodiments 34-37, comprising mixing the blood, prior to said introducing, with one or more autologous cells.
39. The method of embodiment 39, wherein said one or more autologous cells comprise Schwann cell.
40. The method of any one of embodiments 34-39, wherein said one or more coagulating agents and/or anti-anti coagulating agents are in powder form, wherein at least a portion of the particles of said powder are of a size that fits to enter said channels.
41. The method of embodiment 40, comprising filtering said powder prior to mixing it with the blood to obtain a mixture with at least a portion of particles having a size less than 2.5 microns.
42. The method of embodiment 41, wherein said filtering comprises
   introducing the powder into liquid for a selected amount of time,
   collecting floating particles, and
   drying the collected particles to obtain desired filtered particles.

43. The method of any one of embodiments 34-42, wherein said coagulation inducer is an anti-anti-coagulation agent selected from a group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate.
44. The method of any one of embodiments 34-42, wherein said one or more coagulation agents are selected from a group consisting of: Kaolin, Ca 2±, Mg′, negatively charged phospholipid (PL) and protamine sulfate.
45. The method of any one of embodiments 34-43, comprising fitting an enveloping sleeve over at least one of: the first nerve stump and a portion of the nerve graft, the second nerve stump and a portion of the nerve graft or the first nerve stump, the nerve graft and the second nerve stump;
   wherein said introducing comprises filling a lumen of said sleeve with said mixture.
46. A kit for inducing growth of nerve fibers between a first nerve stump and a second nerve stump, the kit comprising:
   a nerve graft having micro channels allowing nerve fibers growth therethrough between the first and second nerve stumps;
   optionally, one or more blood withdrawal devices for withdrawal of blood from the subject;
   one or more blood collection receptacles for receiving the blood;
   a coagulation assembly configured for mixing the withdrawn blood with one or more coagulation inducers for initiating a coagulation process of the withdrawn blood; and
   an applicator for introducing the incomplete coagulated blood into said channels.
47. The kit of embodiment 46, wherein said one or more collection receptacles are vacuum tubes.
48. The kit of embodiment 46 or 47, wherein the coagulation assembly comprises a vessel for receiving the blood, said vessel being configured for contacting the blood with said coagulation inducers.
49. The kit of any one of embodiments 46-48, comprising one or more coagulation inducers.
50. The kit of embodiment 49, wherein the one or more coagulation inducers are comprised within said one or more blood collection receptacles.
51. The kit of embodiment 49 or 50, wherein the coagulation inducers are anti-anticoagulation agent selected from the group consisting of: ACD-A (Anticoagulant Citrate Dextrose, Solution A), EDTA (ethylenediaminetetraacetic Acid), EGTA (ethylene glycol tetraacetic acid), a citrate, Heparin and oxalate.
52. The kit of any one of embodiments 46-51, said one or more coagulation inducers are selected from the group consisting of: Kaolin, Ca′, Mg′, negatively charged phospholipid (PL) and protamine sulfate.
53. The kit of any one of embodiments 46-52, comprising at least one tubular elements for use as a sleeve to be placed over at least one of: the first nerve stump and a portion of the nerve graft, the second nerve stump and a portion of the nerve graft or the first nerve stump, the nerve graft and the second nerve stump.
54. The kit of embodiment 53, wherein said nerve enveloping hollowed element is a nerve guidance conduit.
55. The kit of any one of embodiments 46-54, for use in a method for inducing growth of nerve fibers between a first nerve stump and a second nerve stump, the method comprising:
   (i) placing a nerve graft, comprising microchannels extending therethrough, between the first and second nerve stumps, optionally attaching a first end of the nerve graft to said first nerve stump and a second end of the nerve graft to said second nerve stump;
   (ii) obtaining blood, optionally whole blood withdrawn from the subject;
   (iii) mixing the blood with one or more coagulation inducers;
   (iv) prior to complete coagulation of the blood, introducing the mixture into the grafts microchannels; and
   (v) permitting the blood to coagulate in said microchannels.
56. The kit of any one of embodiments 46-54, for use in a method according to any one of embodiments 34-45.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1D are schematic illustrations exemplifying different stages of the method of inducing growth or regenerating a damaged or cut nerve according to aspects of the present disclosure.

FIGS. 2A-2C are schematic illustrations exemplifying different stages of the method of inducing growth or regenerating a damaged nerve according to an aspect of the present disclosure.

FIG. 3 is a schematic illustration exemplifying an embodiment of the method according to an aspect of the present disclosure.

FIG. 4 is a schematic illustration exemplifying an embodiment of the method according to an aspect of the present disclosure.

FIGS. 5A-5B show test results of treating cut nerve of rabbits with the method of the present disclosure in comparison to cut nerves of rabbits treated by a standard common method. FIG. 5A shows histograms of axon density in the medial portion of the nerve of the two tested groups; FIG. 5B shows cross sections of the medial portion of the nerve of the two tested groups.

### DETAILED DESCRIPTION

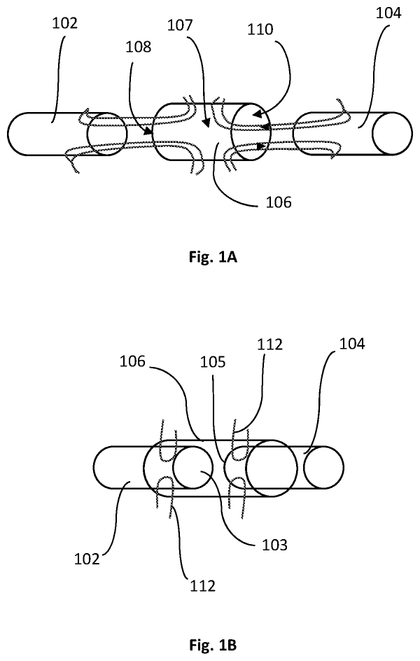

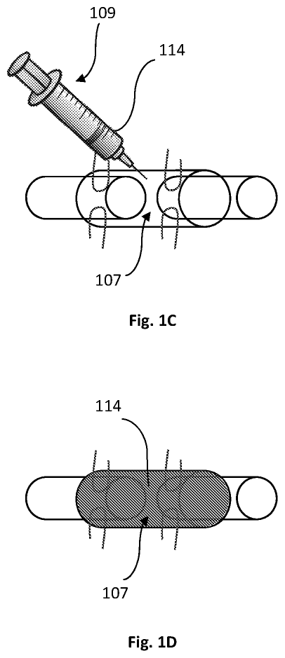

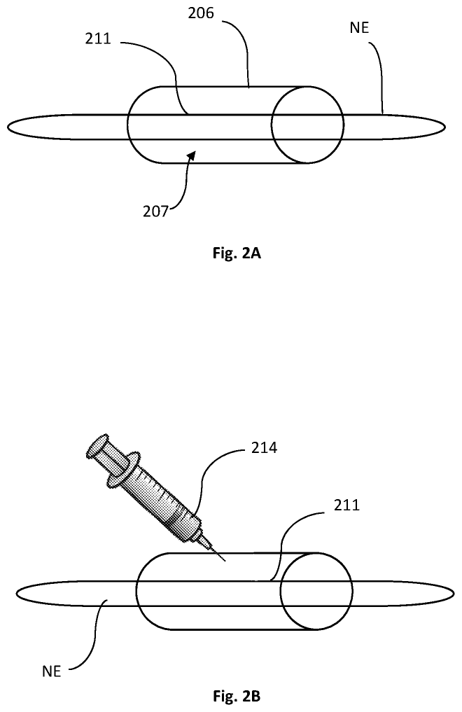

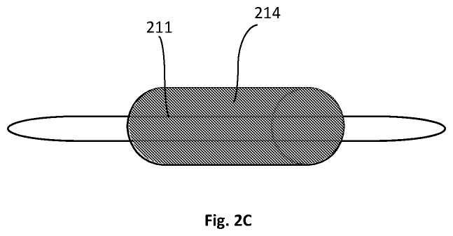

Reference is being made to FIGS. 1A-1D, which are schematic illustrations exemplifying different stages of the method of inducing growth or regenerating a damaged or cut nerve according to aspects of the present disclosure. FIG. 1A shows a first and second stumps 102 and 104 of a cut nerve of a subject being treated. A sleeve 106, e.g. a nerve guidance conduit, is having a lumen 107 extends between first and second open ends 108 and 110 and is configured to receive each of the stumps 102/104 through a respective open end, whereby the sleeve 106 is fitted over the first and second stumps 102 and 104. In FIGS. 1A-1D and FIGS. 2A-2B, the sleeve is presented as transparent to permit view of the elements that would otherwise be concealed thereby, however it is to be noted that the sleeve can be made of materials that have any degree of transparency between full transparency and full opaque.

In FIG. 1A the nerve stumps 102/104 are being introduced via the first and the second open ends 108 and 110, respectively, and being secured, e.g. by suturing to portions of the sleeve that envelopes the stumps. FIG. 1B shows the stumps 102 and 104 within the sleeve 106 and secured in position by securing elements 112 such that a portion that includes the end of each of the stumps 103 and 105 is within the lumen of the sleeve 106 and each of the stumps extends beyond the respective opening.

As can be seen in FIG. 1C, a mixture 114 of whole blood, typically blood withdrawn from the subject, with one or more coagulation inducers, being one or more coagulating agents and/or one or more anti-anti coagulating agents, is introduced into the lumen 107, prior to the complete coagulation of the mixture, namely when the blood is still in a flowable state. The introduction of the mixture into the lumen can be performed by a syringe 109 or any suitable means. The lumen 107 is filled with the mixture such that the mixture contacts at least portions of each of the stumps. FIG. 1D shows that the mixture forms a continuous matrix linking the two stumps 102/104. The coagulation process of the mixture proceeds when it is within the lumen 107 until a clot is formed that contacts or envelopes one or two of the stumps 102/104. The sleeve 106 may be made to fit snugly over the nerve stump. However, in the event this is not the case, in order to retain the mixture within the lumen 107, the first and the second open ends 108 and 110 may be sealed, e.g. by a cloth, a dressing means, or any suitable sealing means.

Reference is now being made to FIGS. 2A-2C, which are schematic illustrations of a non-limiting example of an embodiment according to an aspect of the present disclosure. FIG. 2A shows a sleeve 206 that is fitted over a damaged portion 211 of a nerve NE of a subject. The sleeve may be formed by a sheet wrapped around the nerve NE; or may be formed from a flexible tubular element having an axially extending cut in its wall to permit it to be opened for placing over the nerve. Once the sleeve 206 is in place, a mixture 214 of whole blood, typically blood withdrawn from the subject, with one or more coagulation inducers, is introduced into a lumen 207 of the sleeve 206, prior to the complete coagulation of the mixture, namely when the blood is still in a flowable form, as can be seen in FIG. 2B. The mixture 214 fills the lumen 207 such that it contacts or envelopes the damaged portion 211 of the nerve NE and is maintained in the lumen to form a clot that contacts or envelopes the damaged portion 211 of the nerve NE, as can be seen in FIG. 2C. In time, the clot induces regeneration or growth of the damaged nerve.

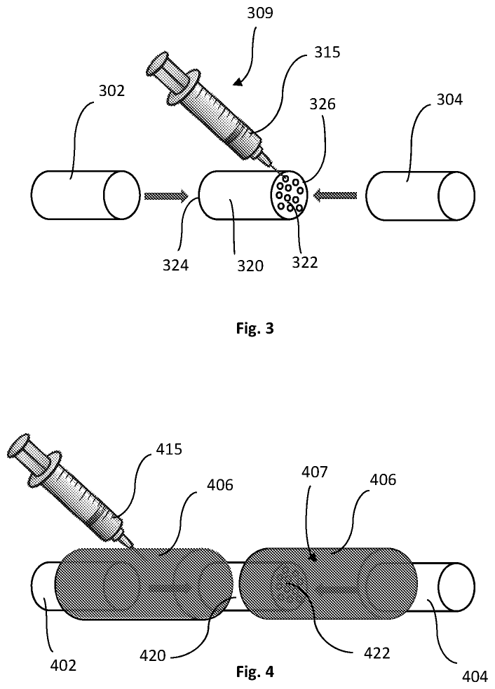

FIG. 3 is a schematic illustration of a non-limiting example of an embodiment of the method according to an aspect of the present disclosure. In this example, the damaged nerve is a cut nerve having a first nerve stump 302 and a second nerve stump 304. The two stumps 302 and 304 are linked to each other via a nerve graft 320 having channels 322 that facilitate nerve growth therethrough. The channels 322 of the nerve graft 320 extending between a first end 324 and a second end 326 of the nerve graft 320. The first end 324 is joined with the first nerve stump 302 and the second end 326 is joined with the second nerve stump 304. A whole blood that, that is typically withdrawn from the subject that is being treated, is mixed with one or more coagulation inducers (namely, coagulation agents and/or anti-anti coagulation agents) to obtain a mixture 315 of whole blood with coagulation inducers, which is then being introduced, by an applicator 309, into the channels 322. The introduction may also be by placing one or both ends 324/326 in the mixture to permit entry of the mixture into the microchannels by capillary forces. The mixture 315 may optionally also be applied in the interfaces between the nerve graft 320 and each of the nerve stumps 302 and 304. The whole blood is then being coagulated in the channels and optional on the interfaces to form clots that enhances the growth of the nerve fibers between the two stumps through the microchannels.

FIG. 4 is a schematic illustration of a non-limiting example of an embodiment of the method according to an aspect of the present disclosure. This example differs from the one in FIG. 3 by enveloping the interfaces between each nerve stump and the nerve graft with a sleeve 406 and optionally securing each of the stumps to the sleeve 406 and/or the nerve graft 420. The lumen 407 of the sleeve 406 is filled with coagulating whole blood such that the channels 422 of the nerve graft 420 are fully or partially filled with the mixture of coagulation initiators and whole blood 415; and also the gap between the stumps 402 and 404 and the nerve graft 420 is filled with coagulating whole blood, which results in a continuous clot between the two stumps.

### Test of the Method of the Present Invention on Rabbits

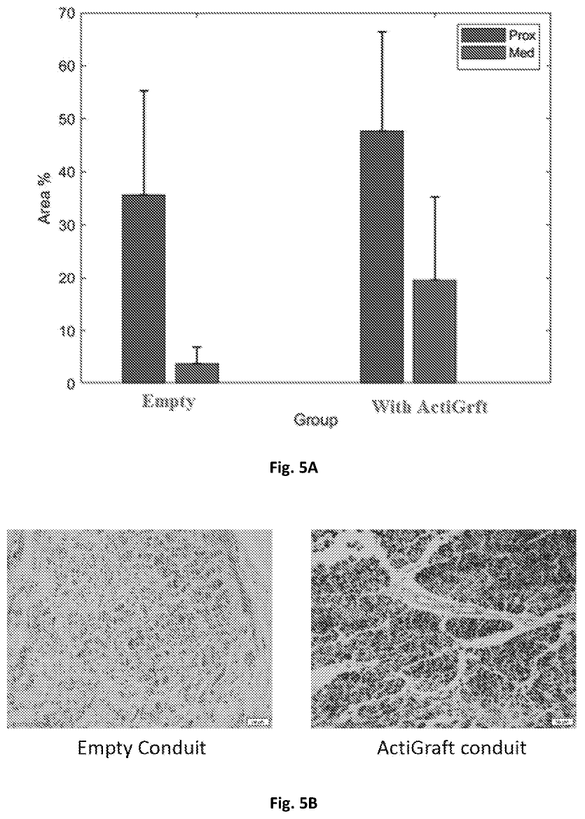

The method of the present disclosure has been tested on a rabbit. A 2.5 cm portion of the Tibial nerve of the rabbit was cut. A nerve guidance conduit was fitted over the two edges of the nerve stump and the gap spanned between them. The two edges were secured to the nerve guidance conduit by suturing. Autologous whole blood was withdrawn from the rabbit and was activated by mixing it with coagulation initiators to initiate blood coagulation. Then, the lumen of the nerve guidance conduit was filled with the activated whole blood of the rabbit to form a blood clot matrix within the lumen that links the two edges. The growth and regeneration status of the nerve of the rabbit was examined 4 months after. FIG. 5A presents histograms of the percentage of axons in a given area of the nerve after 4 months. The left bar of each histogram shows the findings in the proximal portion of the nerve and the right bar of each histogram shows the findings in the medial portion of the nerve. The group that is addressed as “With ActiGrft” represents the rabbit that was tested with the method of the present disclosure. In both portions, the proximal and the medial, more axons were found in the group that used the whole blood clot within the conduit than the group that did not.

FIG. 5B shows cross sections of the medial portion of the cut nerve after 4 months. The left cross section is the control group that did not use the method of the present disclosure, in which the conduit was left empty, and the right cross section is the tested group that used the method of the present disclosure, in which the conduit was filled with coagulated whole blood of the rabbit. The myelin was marked and as can be appreciated, the cross section of the tested group shows much greater axons density than the control group.

1. A kit for inducing growth or regenerating a damaged nerve portion of a subject, the kit comprising:
a nerve enveloping hollow element having a lumen for enveloping said damaged nerve portion such that said portion resides in at least a portion of said lumen;
one or more blood withdrawal devices for allowing withdrawal of blood from the subject;
one or more blood collection receptacles for receiving the blood withdrawn from the subject;
a coagulation assembly configured for permitting mixture of the withdrawn blood with one or more coagulation inducers for initiating coagulation process of the withdrawn blood; and
an applicator for introducing the incomplete coagulated blood into said lumen.

2. The kit of claim 1, wherein the coagulation assembly comprises a volume for introducing the withdrawn blood, said volume is contactable with one or more coagulation agents or anti-anticoagulation agents.

3. The kit of claim 1, comprising one or more anti-coagulation agents for mixing with the withdrawn blood.

4. The kit of claim 1, comprising a film or sheet for placing over said damaged nerve portion for forming said nerve enveloping hollow element.

5. The kit of claim 1, wherein said nerve enveloping hollow element is a nerve guidance conduit.

6. The kit of claim 1, comprising securing elements for securing said damaged nerve portion to the nerve enveloping hollow element.

7. The kit of claim 1, for use in a method for treating, inducing growth, or regenerating a damaged nerve portion, the method comprising:
(i) enveloping said damaged nerve portion with a nerve enveloping hollow element having a lumen such that said damaged nerve portion resides in said lumen;
(ii) withdrawing whole blood from the subject;
(iii) mixing the subject's blood with one or more coagulation inducers;
(iv) prior to complete coagulation of the blood, introducing the blood with the coagulation agent into said lumen; and
(v) permitting the blood to coagulate in said lumen.

8. A method for regenerating a nerve in or across a damaged nerve portion, comprising:
fitting a nerve guidance conduit having a lumen to envelope said nerve portion to reside in said lumen; and
introducing a mixture of whole blood withdrawn from the subject with one or more coagulating inducers into said lumen prior to its complete coagulation.

9. The method of claim 8, wherein said fitting comprises placing a film or sheet over said portion and enveloping said portion with said sheet to form said conduit.

10. A method for inducing growth of nerve fibers across a damaged nerve portion, comprising:
fitting a nerve guidance conduit to envelope said nerve portion; and
introducing a mixture of whole blood withdrawn from the subject with one or more coagulating inducers into said lumen prior to its complete coagulation.

11. The method of claim 8 or 10, wherein the damaged nerve portion comprises cut nerve fibers.

12. The method of claim 11, wherein said nerve portion includes two cut nerve stumps that are surgically joined or brought into proximity.

13. The method of claim 12, wherein said fitting comprises:
placing a film or sheet over said portion and enveloping said portion with said sheet to form said conduit.

14. The method of claim 12, wherein said fitting comprises:
introducing the two nerve stumps into said conduit, one from each end of the conduit; and
bringing the two stumps into proximity to one another within said conduit.

15. The method of claim 14, wherein said bringing, comprises securing the two stumps within said conduit by suturing.

16. The method of claim 10, wherein said introducing comprises filling a portion of the conduit's lumen with the mixture such that it comes into contacts with said nerve portion.

17. The method of claim 16, wherein said nerve portion includes two cut nerve stumps and said introducing is carried out such that said mixture comes into tight contact with said stumps.

18. The method of claim 8 or 10, comprising at least one of: (i) saturating the blood, prior to said introducing, with an oxygen-rich gas; (ii) mixing the blood, prior to said introducing, with one or more autologous cells.

19. The method of claim 18, wherein said one or more autologous cells comprise Schwann cell.

20-32. (canceled)
